(19) United States
(12) Patent Application Publication
(10) Pub. No.: US 2004/0096423 A1
(43) Pub. Date: May 20, 2004

(54) Use of a triple combination comprising a 5ht3 antagonist, a 5ht4agonist and a laxative for promoting intestinal lavage

(76) Inventor: Antonius Adrianus Hendrikus Petrus Megens, Beerse (BE)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/432,811

(22) PCT Filed: Nov. 15, 2001
(86) PCT No.: PCT/EP01/13318

(30) Foreign Application Priority Data
Nov. 24, 2000 (EP) 00204191.1

(51) Int. Cl. A61K 31/765; A61K 31/513; A61K 31/4178

(52) U.S. Cl. 424/78.38; 514/269; 514/397

(57) ABSTRACT

The present invention is concerned with the use of a triple combination comprising a 5HT₃ antagonist, a 5HT₄ agonist and a laxative—in particular an osmotic agent—for accelerating intestinal lavage. The present invention is also concerned with the use of said triple combination for the treatment of constipation.

The present invention is concerned with the use of a triple combination comprising a 5HT₃ antagonist, a 5HT₄ agonist and a laxative—in particular an osmotic agent—for accelerating intestinal lavage. The present invention is also concerned with the use of said triple combination for the treatment of constipation.

Intestinal Lavage

Adequate colon preparation before diagnostic, therapeutic or surgical procedures is important because safety and diagnostic accuracy depend on adequate cleansing of the intestines. Magnesium sulfate (MgSO₄) or, more recently, polyethylene glycol (PEG)-electrolyte solutions (e.g., KleanPrep™ or GoLytely™) have been widely used as lavage solution for colon preparation. These solutions, generally well tolerated by patients, are extremely effective in cleansing the colonic mucosa of faeculent debris. However, fairly large volumes (4 liters) and relatively long preparation times (up to 24 hours) are required. Reduction of the volume to be ingested and shortening of the preparation time would highly increase patient acceptance and comfort.

These agents may also be used to help eliminate parasites following appropriate therapy, for instance these can be used after or in combination with anthelmintics. These osmotic agents may also be used to help eliminate toxic material in some cases of poisoning.

WO-98/47481, published Oct. 29, 1998, discloses the use of a 5HT₃ receptor antagonists in combination with a laxative, in particular an osmotic agent, for accelerating intestinal lavage.

It has now been found that the use of a triple combination comprising a 5HT₃ antagonist, a laxative—in particular an osmotic agent—and a 5HT₄ agonist is even more effective in accelerating intestinal lavage.

Interesting compounds having 5HT₃ antagonistic properties are alosetron, azasetron, cilansetron, dolasetron, granisetron, indisetron, itasetron, lerisetron, lurosetron, ondansetron, R-ondansetron, S-ondansetron, palonosetron, ramosetron, tropisetron, ((−)-cis-4-amino-5-chloro-2,3-dihydro-N-[1-[3-[(3,4-dihydro-4-oxo-2-pyrimidinyl)-amino]propyl]-3-methoxy-4-piperidinyl]-2,2-dimethyl-7-benzofurancarboxamide), which will be referred to hereinafter as “COMPOUND A”, and alike compounds.

Interesting compounds having 5HT₄ agonistic properties are cisapride, prucalopride, mosapride, renzapride, tegaserod, E3620, and alike compounds.

Another interesting 5HT₄ agonist is (3S-trans)-4-amino-5-chloro-2,3-dihydro-N-[[3-hydroxy-1-(3-methoxypropyl)-4-piperidinyl]methyl]-2,2-dimethyl-7-benzofuran-carboxamide, which will be referred to hereinafter as “COMPOUND B”, which is described as compound number 95 of WO 99/02156, published on 21 Jan. 1999.

Of course the pharmaceutically acceptable acid or base addition salt of the 5HT₃ antagonists and 5HT₄ agonists are also intended to be included in the present invention. The pharmaceutically acceptable acid addition salts as mentioned hereinabove are meant to comprise the therapeutically active non-toxic acid addition salt forms which the 5HT₃ antagonists and the 5HT₄ agonists are able to form. The latter can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

Those 5HT₃ antagonists and 5HT₄ agonists containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term addition salt as used hereinabove also comprises the solvates which the compounds of formula (I) as well as the salts thereof, are able to form. Such solvates are for example hydrates, alcoholates and the like.

Laxatives are drugs that promote defecation. Precise mechanisms of action of many laxatives remain uncertain because of the complex factors that affect colonic function, prominent variations of water and electrolyte transport among experimental species and preparations, and a certain expensiveness of research in this area. Three general mechanisms of laxative action can be described. (1) By their hydrophilic or osmotic properties, laxatives may cause retention of fluid in colonic contents, thereby increasing bulk and softness and facilitating transit. (2) Laxatives may act, both directly and indirectly, on the colonic mucosa to decrease net absorption of water and NaCl. (3) Laxatives may increase intestinal motility, causing decreased absorption of salt and water secondary to decreased transit time. Mostly one recognizes three major classes of laxatives, i.e. 1) dietary fiber and bulk-forming laxatives, 2) saline and osmotic laxatives and 3) stimulant laxatives. (see Goodman and Gilman, seventh edition, pp 994 to 1003).

The bulk-forming laxatives include a wide range of natural and semisynthetic polysaccharides and cellular derivatives that are only partially digested. The undigested portions are hydrophilic and swell in the presence of water to form a viscous solution or gel. The increased intraluminal pressure reflexively stimulates peristalsis, diminishes colonic transit time and produces a soft gelatinous stool (“Remington's Pharmaceutical Sciences”, page 783-786, 1990, Mack Publishing Company, Easton, Pa., 18th edition).

The stimulant laxatives act on the intestinal tract to increase its motor activity. The more commonly employed agents are the anthraquinone laxatives, such as, e.g. cascara sagrada and senna; the diphenylmethane derivatives, such as, e.g. phenolphtalein and bisacodyl; and castor oil (“Remington's Pharmaceutical Sciences”, page 783-786, 1990, Mack Publishing Company, Easton, Pa., 18th edition).

Saline and osmotic laxatives are the primary class of laxatives envisaged in this invention.

Saline and osmotic laxatives include various magnesium salts; the sulfate, phosphate, and tartrate salts of sodium and potassium; the dissacharide lactulose; glycerin; and sorbitol. They are poorly and slowly absorbed and act by their osmotic properties in the luminal fluid.

Two examples of these osmotic agents which are commercially available for intestinal cleansing are KleanPrep™ and GoLytely™.

5HT₃-receptor antagonists can be identified by the fact that they are active, for example, in antagonising the Von Bezold-Jarisch chemoreflex evoked by serotonin in rats (Pharmacology and Toxicology, 70, Supp II, 17-22 (1992)). An in vitro binding assay to measure the Kᵢ value for 5HT₃ receptor binding is described in Pharmacological Example 1.

An in vitro binding assay to measure the EC₅₀ value for 5HT₄ receptor binding is described in Pharmacological Example 2.

The present invention is concerned with the use of a triple combination comprising a 5HT₃ antagonist, a 5HT₄ agonist and a laxative—in particular an osmotic agent—for the manufacture of a medicament for accelerating or promoting intestinal lavage. Hence, a method of treatment is claimed whereby an effective amount of a 5HT₃ antagonist and a 5HT₄ agonist is administered to a warm-blooded animal, in particular a mammal, in combination with a laxative, in particular an osmotic agent.

The 5HT₃ antagonism and 5HT₄ agonism might also be combined in one and the same compound.

The terms “accelerating”, “improving”, or “promoting” are used as synonyms throughout this text.

The patients envisaged in this treatment are people whose bowel needs to be cleaned prior to diagnostic or surgical procedures. Another group of patients are those patients who are to be prevented from straining at the stool, these patients include people suffering from hernia or cardiovascular disease. In addition, the combination of the present invention can be indicated, both before and after surgery, to maintain soft feces in patients with hemorrhoids and other anorectal disorders.

Osmotic agents at cathartic doses are frequently employed prior to radiological examination of the gastrointestinal tract, kidneys, or other abdominal or retroperitoneal structures and prior to elective bowel surgery. Hence, also for these applications the presently described combination may be useful.

Furthermore the combination of the present invention can also be used in the treatment of drug overdosage and poisoning, by removing agents from the intestine. The combination of the present invention may also be employed in further combination with with certain anthelmintics.

As is demonstrated in the experimental part the present invention provides a method to accelerate and/or enforce the action of laxatives, especially osmotic agents. The laxatives can be administered or co-administered orally or rectally. Also provided is a method of accelerating intestinal lavage in a warm-blooded animal, in particular a mammal, by administration of a laxative in combination with an effective amount of a 5HT₃ antagonist and a 5HT₄ agonist.

In general, “co-administration” means that the laxative, the 5HT₃ antagonist and the 5HT₄ agonist are present in the gastro-intestinal tract during at least partially overlapping times. Additionally, “co-administration” comprehends administering more than one dose of said laxative within 1 hour after a dose of the 5HT₃ antagonist and the 5HT₄ agonist, in other words, the 5HT₃ antagonist and the 5HT₄ agonist need not be administered again before or with every administration of said laxative, but may be administered intermittently during the course of treatment.

The present invention is also concerned with the use of a triple combination comprising a 5HT₃ antagonist, a 5HT₄ agonist and a laxative—in particular an osmotic agent—for the manufacture of a medicament for the treatment of constipation, such as acute constipation, chronic constipation or refractory constipation. Consequently, a method is provided to treat constipation, such as, e.g. acute constipation, chronic constipation or refractory constipation, in warm-blooded animals, in particular mammals, by administration of a laxative in combination with an effective amount of a 5HT₃ antagonist and an effective amount of a 5HT₄ agonist.

The 5HT₃ antagonists and the 5HT₄ agonists may be formulated into various pharmaceutical forms for administration purposes. To prepare these pharmaceutical compositions, an effective amount of a particular compound, in base or acid addition salt form, as the active ingredient is intimately mixed with a pharmaceutically acceptable carrier. Said carrier may take a wide variety of forms depending on the form of preparation desired for administration. These pharmaceutical compositions are desirably in unitary dosage form suitable, preferably, for administration orally, rectally or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect to the skin. Said additives may facilitate the administration to the skin and/or may be helpful for preparing the desired compositions. These compositions may be administered in various ways, e.g., as a transdermal patch, as a spot-on, as an ointment. Acid addition salts of the compounds of formula (I) due to their increased water solubility over the corresponding base form, are obviously more suitable in the preparation of aqueous compositions. It is especially advantageous to formulate the aforementioned pharmaceutical compositions in dosage unit form for ease of administration and uniformity of dosage. Dosage unit form as used in the specification herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The dosages of the drugs used in the present invention must, in the final analysis, be set by the physician in charge of the case, using knowledge of the drugs, the properties of the drugs in combination as determined in clinical trials, and the characteristics of the patient, including diseases other than that for which the physician is treating the patient.

In general it is contemplated that an effective amount of a 5HT₃ antagonist would be from about 0.001 mg/kg to about 50 mg/kg body weight, preferably from about 0.02 mg/kg to about 5 mg/kg body weight. An effective amount of a 5HT₄ agonist would be from about 0.001 mg/kg to about 50 mg/kg body weight, preferably from about 0.02 mg/kg to about 5 mg/kg body weight. Precise dosage rates and regimes can be determined empirically by the medical practitioner, depending on individual circumstances.

In an embodiment the amount of the 5HT₃ antagonist ranges from 0.001 mg/kg to 0.1 mg/kg, preferably about 0.01 mg/kg, and the amount of the 5HT₄ agonist ranges from 0.001 mg/kg to 1 mg/kg.

As an additional feature of the invention, this invention provides a therapeutic package suitable for commercial sale, comprising a container, an dosage form of a 5HT₃ antagonist, a 5HT₄ agonist and a laxative, in particular an osmotic agent. This laxative or osmotic agent is often in the form of a powder, which is normally to be dissolved or suspended in a certain amount of water. Consequently, the present invention also relates to a product comprising a 5HT₃ antagonist, a 5HT₄ agonist and a laxative, in particular an osmotic agent, for simultaneous or sequential use in the treatment of constipation or for promoting intestinal lavage, provided that said product does not contain an opioid antagonist. The amount of each component, i.e. a 5HT₃ antagonist, a 5HT₄ agonist and a laxative, in said product is such that the combination of the three components exhibits a synergistic effect. Such a product may comprise a kit comprising a container containing a pharmaceutical composition of a laxative, another container comprising a pharmaceutical composition of the 5HT₃ antagonist, and another container comprising the 5HT₄ agonist. The product with separate compositions of the laxative, the 5HT₃ antagonist and the 5HT₄ agonist has the advantage that appropriate amounts of each component, and timing and sequence of administration can be selected in function of the patient.

### PHARMACOLOGICAL EXAMPLES

1. In Vitro Binding Affinity for the 5HT₃ Receptor

In vitro 5HT₃ receptor binding was measured using N×G 108CC15 cells and [³H]GR 65630. Cells were homogenized in tris.HCl buffer (20 mM, pH=7.5) containing NaCl (154 mM); the final cell concentration corresponded to approximately 10⁶ cell/ml. Incubation mixtures for radioligand binding assays were composed of 0.5 ml membrane suspensions, 0.025 ml [³H]GR 65630 (final concentration 2 nM), and 0.025 ml either solvent (10% dimethylsulfoxide) for total binding, or tropisetron (final concentration 1.0 μM) for non-specific binding, or drug solution. The incubation was run for 60 minutes at 37° C. Labelled membranes were collected and raised by rapid filtration under suction over Walkman GF/B plan fibre filters (presoaked in 0.1% polyethyleneimine for at least 1 hour) using a 40-well filtration manifold. Test compounds were added at appropriate concentrations (60 minutes incubation time), in such a way that the inhibition curves were defined by at least eight to twelve concentration points, measured in duplicate. All experiments were repeated independently at least two times. Radioactivity on the filters was counted in a Packard Tri-carb 1600CA liquid scintillation analyzer. Counting data were collected directly in a Macintosh SE personal computer and further transferred to a Macintosh II personal computer. Counting data from assays in the presence of a compound were automatically expressed as percent of total binding measured in the absence of test compound. Inhibition curves plotting percent of total binding versus the log concentrations of the test compound were automatically generated. The sigmoidal inhibition curves were analyzed by computerized curve-fitting, with a programme using non-linear regression analysis for one- or two site curve fitting (modifications of equations described in Oestreicher E. G. and Pinto G. F., Comput. Biol. Med., 17, 53-68 (1987). The -log IC₅₀ values (pIC₅₀; IC₅₀ defined as the concentration producing 50% inhibition of specific radioligand binding) were derived from individual curves. Kᵢ values were calculated according to the method of Cheng and Prusoff (Cheng Y. C. and Prusoff W. H., Biochem. Pharmacol., 22, 3099-3108, 1973)[Kᵢ=IC₅₀/(1+C/K_{D})] using the K_{D} (1.7 nM) and the concentration (C) of the radiolabelled [³H]GR 65630 (2 nM). The Kᵢ values of the test compounds are presented as logarithmic mean and corresponding 95% confidence limits of the various determinations; and are presented in Table 1 in the column labelled “5HT₃ receptor Kᵢ (95% c.l.) nM”.

TABLE 1
Binding to the serotonin 5HT₃ receptor (Kᵢ, nM).

| | 5HT₃ receptor |
|---|---|
| Test compound | Kᵢ (95% c.l.) nM |
| bemesetron | 8.0 (2.2-29) |
| granisetron | 2.6 (1.2-5.7) |
| ondansetron | 8.4 (6.2-11) |
| renzapride | 10 (4.4-23) |
| tropisetron | 0.78 (0.35-1.7) |
| zacopride | 0.46 (0.34-0.62) |
| Compound A | 0.58 (0.10-3.4) |

### EXAMPLE 2

### “In Vitro Binding Affinity for the 5HT₄ Receptor”

Dunkin-Hartley guinea-pigs of either sex (weighing between 600-900 g) were killed by decapitation. The ileum was removed and cleansed with warmed and oxygenated Krebs-Henseleit solution. Parts of the ileum (15 cm) were slipped over a glass pipette. The longitudinal muscle layer with myenteric plexus was removed by means of a cotton thread moistened with Krebs solution. Strips with a length of 8 cm were folded and these strips (4 cm) were mounted between two platinum electrodes (8 cm length, 0.5 cm apart). The strips were suspended with a preload of 1.5 g in 100 ml Krebs-Henseleit solution (37.5° C.), gassed with a mixture of 95% O₂ and 5% CO₂. The preparations were excited with single rectangular stimuli [1 ms; 0.1 Hz; submaximal response (current leading to 80% of maximal response), from a programmable stimulator (Janssen Scientific Instruments Division)]. Contractions were measured isometrically (Statham UC2, Janssen Scientific Instruments amplifier, Kipp BD-9 pen-recorder). During the stabilization period of 30 minutes, the strips were repeatedly stretched, in order to obtain a steady state tension of 1.5 g. Before starting the electrical stimulation, a cumulative concentration response curve of acetylcholine (3.10⁻⁹, 10⁻⁸, 3.10⁻⁸ and 10⁻⁷ M) was given. The bath fluid was replaced with fresh Krebs solution and the strips were allowed to stabilize for another 30 minutes. Subsequently, the strips were stimulated electrically (power stimulator) at a frequency of 0.1 Hz for 1 ms. The voltage was increased by steps of 2 V (maximum 15 V) until maximum force development was observed. The twitch response was decreased (by voltage reduction) to about 80% of that operative at maximal voltage. By adjusting the voltage carefully it was possible to obtain a submaximal twitch response which did not vary over at least 2 hours. When the twitch responses were stable for at least 15 minutes, the test compound was added to the bath fluid for 30 minutes. If the test compound caused less than 50% inhibition, cisapride 3.10⁻⁷ M was added to the bath fluid to find out whether the test compound could antagonize the stimulatory effect of cisapride. If the test compound caused more than 50% inhibition, naloxone 10⁻⁷ M was added to find out whether the inhibition was mediated via opiate receptors. After the addition of either cisapride or naloxone, a supramaximal stimulation was given again. Afterwards, the electrical stimulation was discontinued and a second cumulative concentration-response curve with acetylcholine was given. These two cumulative concentration-response curves of acetylcholine were given in order to distinguish effects via a decreased acetylcholine release, from a direct anticholinergic effect or to distinguish effects via enhanced release of acetylcholine from sensitization of muscarinic receptors. The EC₅₀ (i.e. the concentration that stimulates the response to electrical stimulation by 50%) of a compound, was calculated using linear regression analysis when the test compound causes stimulation.

TABLE 2
EC₅₀ values (nM)

| Test compound | EC₅₀ (nM) |
|---|---|
| cisapride | 63 |
| prucalopride | 16 |
| Compound B | 1.6 |

### EXAMPLE 4

### Measurement of Onset of Liquid Stool

1) General Description.

The dogs were orally or subcutaneously pretreated with test compounds or distilled water (0.5 ml/kg) and 1 hour later challenged by gavage with KleanPrep™ (standard volume: 2×200 ml at a 15 minutes interval). The time interval at which the first liquid stool occurred was noted (in minutes after the first administration of KleanPrep™) up to 6 hours after challenge. By empirical observation there was no interference of urinary excretion: urinating dogs did not show liquid stools (rather the reverse was found: dogs without liquid stools always urinated) and dogs with liquid stools did never urinate.

The KleanPrep™ preparation consisted of polyethyleneglycol 3350 (59.000 g/l), sodium sulphate (5.685 g/l), sodium hydrogencarbonate (1.685 g/l), sodium chloride (1.465 g/l), potassium chloride (0.7425 g/l), aspartate (0.0494 g/l) and vanilla (0.3291 g/l).

2) Statistics

Each dose of the test compounds (the SHT₃ antagonist Compound A, and the 5HT₄ agonists cisapride, prucalopride and Compound B) was given to 5 animals. All-or-none criteria were used to calculate ED₅₀-values and 95% confidence limits according to the iterative method of Finney.

3) Control Data Obtained using the Osmotic Agent KleanPrep™

In a pilot study on five dogs, large volumes of KleanPrep™ (1400, 1400, 1800, 2800, 2800 ml) and relatively long time intervals (95, 101, 124, 211 and 305 minutes after KleanPrep™) were required to obtain liquid stools within an observation period of 6 hours. In the present experiments, a standard volume of 400 ml KleanPrep™ solution (2×200 ml at a 15 minutes interval) was used. Under these conditions, liquid stools were not observed up to 6 hours after KleanPrep™, neither in 40 control dogs pretreated with a single dose of distilled water (0.5 ml/kg, p.o., −1 hour), nor in 35 control dogs pretreated with two doses of distilled water (0.5 ml/kg, p.o., −1 hour) immediately after each other, nor in 10 dogs pretreated with a dose of distilled water (0.5 ml/kg, p.o., −1 hour) and a dose of saline (0.5 ml/kg, s.c., −1 hour), nor in 5 dogs treated with two doses of distilled water (0.5 ml/kg, p.o., −1 hour) and a dose of saline (0.5 ml/kg, s.c., −1 hour).

4) Effect of the 5HT₄ Agonist Cisapride

In order to obtain liquid stools within 6 hours after administration of KleanPrep™, an ED₅₀ of 0.89 mg/kg was required.

5) Effect of the 5HT₄ Agonist Prucalopride

Prucalopride was not very potent in terms of the dose required for obtaining liquid stools within 6 hours (ED₅₀:2.3 mg/kg) after administration of KleanPrep™. Liquid stools within 1 hour were not obtained up to 5 mg/kg.

6) Effect of the 5HT₃ Agonist Compound B

Compound B did not induce liquid stools after administration of KleanPrep™ over the 6 hour observation period up to the dose of 2.5 mg/kg.

7) Effect of the Triple Combinations

7.1 Control Data Obtained with the 5HT₃ Antagonist Compound A

Thirty-five control dogs received KleanPrep™ 1 hour after combined pre-treatment with the 5HT₃ antagonist Compound A (0.01 mg/kg, p.o.) and distilled water (0.5 ml/kg, p.o.). Thirty-three of them displayed liquid stools within 6 hours (median time interval over the 35 animals: 125 minutes) but only two displayed liquid stools within 1 hour after administration of KleanPrep™.

7.2) Combination of the 5HT₄ Agonist Cisapride with the 5HT₃ Antagonist Compound A

In dogs pretreated with Compound A (0.01 mg/kg), co-administration of the 5HT₄ agonist cisapride dose-dependently accelerated the onset of the first liquid stool from a median of 125 minutes in the control animals down to 24 minutes after administration of KleanPrep™ (ED₅₀ for liquid stools within 1 hour: 0.056 mg/kg).

7.3) Combination of the 5HT₄ Agonist Prucalopride with the 5HT₃ Antagonist Compound A

In dogs pretreated with Compound A (0.01 mg/kg), co-administration of the 5HT₄ agonist prucalopride dose-dependently accelerated the onset of the first liquid stool from a median of 125 minutes in the control animals down to 32 minutes after administration of KleanPrep™ (ED₅₀ for liquid stools within 1 hour: 0.22 mg/kg).

7.4) Combination of the 5HT₄ Agonist Compound B with the 5HT₃ Antagonist Compound A

In dogs pretreated with Compound A (0.01 mg/kg), co-administration of the 5HT₄ agonist Compound B dose-dependently accelerated the onset of the first liquid stool from a median of 125 minutes in the control animals down to 29 minutes after administration of KleanPrep™ (ED₅₀ for liquid stools within 1 hour: 0.0014 mg/kg).

TABLE 3
median onset of the first liquid stool after
administration of KleanPrep ™.

| | osmotic agent + 5HT₃ antagonist (Compound A) | | | |
|---|---|---|---|---|
| 5HT₄ agonist | none (control) | cisapride | prucalopride | Compound B |
| median onset | 125 | 24 | 32 | 29 |
| (minutes) | | | | |

1. Use of a triple combination comprising a 5HT₃ antagonist, a 5HT₄ agonist and a laxative for the manufacture of a medicament for accelerating intestinal lavage.

2. Use of a triple combination comprising a 5HT₃ antagonist, a 5HT₄ agonist and a laxative for the manufacture of a medicament for treating constipation.

3. Use according to claims 1 to 2 wherein the laxative is an osmotic agent.

4. Use according to claims 1 to 3 wherein the laxative is a polyethylene glycol (PEG)-electrolyte solution.

5. Use according to any of the above claims wherein the 5HT₃ antagonist is selected from the group consisting of alosetron, azasetron, cilansetron, dolasetron, granisetron, indisetron, itasetron, lerisetron, lurosetron, ondansetron, R-ondansetron, S-ondansetron, palonosetron, ramosetron, tropisetron, (−)-cis-4-amino-5-chloro-2,3-dihydro-N-[1-[3-[(3,4-dihydro-4-oxo-2-pyrimidinyl)amino]propyl]-3-methoxy-4-piperidinyl]-2,2-dimethyl-7-benzofurancarboxamide and the pharmaceutically acceptable acid addition salts thereof.

6. Use according to claim 5 wherein the 5HT₃ antagonist is (−)-cis-4-amino-5-chloro-2,3-dihydro-N-[1-[3-[(3,4-dihydro-4-oxo-2-pyrimidinyl)amino]-propyl]3-methoxy-4-piperidinyl]-2,2-dimethyl-7-benzofuran-carboxamide.

7. Use according to any of the above claims wherein the 5HT₄ agonist is selected from the group consisting of cisapride, prucalopride, mosapride, tegaserod or (3S-trans)-4-amino-5-chloro-2,3-dihydro-N-[[3-hydroxy-1-(3-methoxypropyl)-4-piperidinyl]-methyl]-2,2-dimethyl-7-benzofuran-carboxamide.

8. Use according to claim 7 wherein the 5HT₄ agonist is (3S-trans)-4-amino-5-chloro-2,3-dihydro-N-[[3-hydroxy-1-(3-methoxypropyl)-4-piperidinyl]methyl]-2,2-dimethyl-7-benzofuran-carboxamide.

9. Use according to any of claims 1 to 4 wherein the amount of the 5HT₃ antagonist ranges from 0.001 mg/kg to 0.1 mg/kg and the amount of the 5HT₄ agonist ranges from 0.001 mg/kg to 1 mg/kg.

10. Use according to claim 9 wherein the amount of the 5HT₃ antagonist is 0.01 mg/kg and the amount of the 5HT₄ agonist ranges from 0.001 mg/kg to 1 mg/kg.
